(19) United States
(12) Patent Application Publication
(10) Pub. No.: US 2026/0157852 A1
(43) Pub. Date: Jun. 11, 2026

(54) IMPLANTS INCLUDING MODIFIED DEMINERALIZED CORTICAL BONE FIBERS AND METHODS OF MAKING SAME

(71) Applicant: MUSCULOSKELETAL TRANSPLANT FOUNDATION, Edison, NJ (US)

(72) Inventors: Michele Christine McAllister, Freehold, NJ (US); David Wang, Rockville, MD (US); Michelle Bubear, Newton, NJ (US); Joed Canales, Harrison, NJ (US); Amy Chang, Kaneohe, HI (US); Roman Shikhanovich, Edison, NJ (US); Mark Spilker, Plainsboro, NJ (US); Eric Semler, Morganville, NJ (US)

(21) Appl. No.: 19/181,529

(22) Filed: Apr. 17, 2025

Related U.S. Application Data
(62) Division of application No. 17/966,092, filed on Oct. 14, 2022, now Pat. No. 12,295,848.
Application No. 17/966,092 is a continuation of application No. 16/598,354, filed on Oct. 10, 2019, now Pat. No. 11,596,517.
Application No. 16/598,354 is a division of application No. 15/159,406, filed on May 19, 2016, now Pat. No. 10,531,957.
(60) Provisional application No. 62/164,827, filed on May 21, 2015.
(60) Provisional application No. 62/331,071, filed on May 3, 2016.

(51) Int. Cl. A61F 2/28; A61L 27/36

(52) U.S. Cl. CPC A61F 2/28; A61L 27/3604; A61L 27/3608; A61L 27/365; A61L 27/3683; A61F 2240/001; A61L 2400/18; A61L 2430/02; A61L 2430/38

(57) ABSTRACT

Methods for making surgical implants (or grafts) for the repair of bone defects, and more particularly, surgical implants that include demineralized bone fibers and mineralized bone pieces, are disclosed. Also disclosed are methods for increasing the wettability and ensuring uniform density of such implants. The surgical implants have a wettability time of less than 5 minutes, a residual moisture content of less than 6% by weight, and a complete rehydration time of less than about 30 minutes. The implants remain cohesive and retain their shape upon complete rehydration, and may be reshaped to a second shape which is retained until reshaped by external force.

### CROSS-REFERENCE TO RELATED APPLICATIONS

This application is a divisional of U.S. patent application Ser. No. 17/966,092, filed Oct. 14, 2022, now allowed and which is a continuation of U.S. patent application Ser. No. 16/598,354, filed Oct. 10, 2019, which issued as U.S. Pat. No. 11,596,517 on Mar. 7, 2023 and which is a divisional of U.S. patent application Ser. No. 15/159,406 filed on May 19, 2016, which issued as U.S. Pat. No. 10,531,957 on Jan. 14, 2020 and which claims the benefit of U.S. Provisional Application No. 62/331,071, filed May 3, 2016, and U.S. Provisional Application No. 62/164,827, filed May 21, 2015, the entire disclosures of all of which are incorporated by reference herein. This application also relates to commonly owned International Application No. PCT/US16/33246, filed May 19, 2016 and entitled “Modified Demineralized Cortical Bone Fibers,” the entire disclosure of which is incorporated by reference herein.

### FIELD OF THE INVENTION

The disclosed invention relates to the field of surgical grafts for the repair of bone defects, more particularly, surgical grafts that include demineralized bone particles.

### BACKGROUND OF THE INVENTION

Cohesive masses of demineralized cortical bone fibers have been used as bone void fillers or implants for use in general orthopaedic applications, trauma applications, and spinal applications, as well as for repair of craniomaxial defects, dental defects, and other bony defects. Such bone void fillers and implants absorb liquids, such as saline, blood, or bone marrow aspirate, but are slow to wet upon initial contact with a liquid. Further, the hydrated mass of fibers in such implants tends to lack structural strength such that it breaks apart when manipulated or irrigated.

### SUMMARY OF THE INVENTION

Demineralized cortical bone fibers may be modified to improve certain properties of cohesive masses of such fibers that affect their usefulness as surgical grafts for bone repair. Such properties include wettability (i.e., surface tension or hydrophilicity), structural stability after compression, reduced swelling upon hydration, resistance to wash-out of fibers during irrigation, and ease of molding the fiber masses in their hydrated form. In a process according to an embodiment of the present invention, the wettability of the demineralized cortical bone fibers is increased by treating them with a biocompatible polar molecule. In an embodiment, the polar molecule comprises one or more of an alcohol, a polyol (e.g., a glycol or a glycerol), a sugar, a ketone, an aldehyde, an organic acid, or another biocompatible polar organic compound. In a process according to an embodiment of the present invention, the wettability of the demineralized cortical bone fibers is increased by treating them with a salt solution, such as saline solution or phosphate buffer. In a process according to an embodiment of the present invention, the wettability of the demineralized cortical bone fibers and/or masses of cortical bone fibers are modified by exposing them to an energetic source such as ultraviolet (UV) radiation. Embodiments of the present invention also include demineralized cortical bone fibers prepared by the aforementioned processes, masses of such demineralized cortical bone fibers, and surgical grafts and implants that include such demineralized cortical bone fibers.

Other embodiments of the present invention include chemical cross-linking of the demineralized cortical bone fibers. Still other embodiments include modifying the surface tension of the fibers by increasing their surface roughness or by drying at least one surface of the implant in contact with an appropriate solid or mesh material.

In embodiments of the present invention, any of the aforesaid methods may be used to treat other forms of demineralized bone matrix, such as demineralized cancellous bone pieces, demineralized cortical bone pieces, or fragments of demineralized bone. The aforesaid methods may also be used to increase the wettability of fibers or other graft materials that include tissue types derived from suitable organs or other tissue sources, or the wettability and/or mechanical properties of masses of such tissue particles.

Embodiments of the present invention include UV containment chambers which enable optimal exposure of the implant to UV radiation, while protecting an operator from exposure to potentially harmful UV radiation. Such containment chambers are specially designed for specific embodiments of the energetic cross-linking process.

### BRIEF DESCRIPTION OF THE FIGURES

For a more complete understanding of the present invention, reference is made to the following detailed description of exemplary embodiments considered in conjunction with the accompanying figures, in which:

FIG. 1 is a block diagram of a process for modifying demineralized bone particles by a chemical treatment according to an embodiment of the present invention;

FIG. 2 is a block diagram of a process for modifying demineralized bone particles by exposure to ultraviolet radiation according to an embodiment of the present invention;

FIGS. 3A-3C are schematic partial cross-sectional views of a rectangular syringe mold as used in one exemplary embodiment of a process for making implants from demineralized cortical bone fiber;

FIG. 4 is an image showing demineralized cortical bone fiber implants with varying degrees of radio-opacity imparted by the addition of mineralized cortical bone

FIG. 5 is a block diagram of a process for modifying demineralized bone particles by a chemical treatment and a curing step according to another embodiment of the present invention; and

FIGS. 6A-6C are schematic partial cross-sectional views of a rectangular syringe mold having a plunger with perforation which is used in one exemplary embodiment of a process for making implants from demineralized cortical bone fiber.

### DETAILED DESCRIPTION OF THE INVENTION

Embodiments of the present invention include methods of treating demineralized bone particles to increase the wettability (i.e., surface tension or hydrophilicity) of the particles and modify the wettability and structural properties of implants including such particles. Although the exemplary embodiments presented herein describe the treatment of demineralized cortical bone fibers, the methods may be extended to the treatment of other demineralized bone matrix particles, such as demineralized cortical bone pieces, demineralized cancellous bone pieces, or corticocancellous bone pieces. The methods discussed herein may also be used to treat particles and implants derived from other tissue types. It is noted that the demineralized bone matrix particles and/or other tissue types may be used to make autografts, allografts or xenografts. All such options are within the contemplation of the methods and articles described hereinafter.

### I. DEMINERALIZED BONE MATRIX PARTICLES AND IMPLANTS COMPRISING SUCH PARTICLES

“Demineralized bone matrix” (DBM) refers to a bone-derived material that has osteoconductive and osteoinductive activity. DBM may be prepared by acid extraction of allograft bone, resulting in loss of most of the mineralized component but retention of collagen and noncollagenous proteins, including growth factors. Calcium can also be extracted from bone using such compounds as guanidine, ethylenediaminetetraacetatic acid (EDTA), urea, or other compounds that can form soluble complexes with calcium. DBM can be prepared in batch processes (e.g., in a flask, beaker, or other container), by a static or agitated soak, or in a flow-through apparatus whereby the bone is maintained in the apparatus while the demineralizing solution flows through. In agitated soaks, the bone is agitated in the demineralizing solution using methods that employ shaking, stirring, vibration, or ultrasonic techniques. Methods for preparing demineralized bone matrix from bone are known in the art, as disclosed, for example, in U.S. Pat. Nos. 5,073,373; 5,484,601; and 5,284,655, which are incorporated by reference herein. DBM may be prepared from autologous bone, allogeneic (or “allograft”) bone, or xenogeneic bone. DBM may be prepared from cancellous bone, cortical bone, corticocancellous bone, or combinations of cancellous, cortical and corticocancellous bone.

“Demineralized cortical bone fibers” (“DCBF”) refers to elongated particles of DBM derived from cortical bone, which have a length that is at least twice as great as the thickness and width of the fiber. Elongated particles of other tissue types discussed in this disclosure are also “fibers” for the purpose of this disclosure when they have respective lengths that are at least twice as great as their respective thicknesses and widths.

DCBF, according to embodiments of the present invention, may be derived from the cortical component of the long bones of the femur, tibia, humerus, radius, ulna, and fibula, or other suitable long bones of a mammal. Suitable mammal sources for DCBF include, without limitation, human, bovine, ovine, caprine, and porcine sources. The cortical bone is first stripped of all soft tissue elements and then cleaned using detergents/surfactants to remove residual blood and lipids from the bone surface. The cleaned cortical bone is then processed into elongated particles using a milling process that results in fibers that range in size from about 10 μm to about 1000 μm in thickness, about 20 μm to about 20 cm in length and about 5 μm to about 1 cm in width. The cortical fibers are demineralized in dilute acid resulting in a residual calcium content ranging from less than 15% w/w for partially demineralized fibers, less than 8% w/w for demineralized fibers, and less than 1% w/w for substantially or fully demineralized fibers. The calcium content of the fully demineralized fibers may be negligibly small, such that the fibers consist essentially of collagen, non-collagen protein, including glycoproteins, growth factors, and other non-mineral substances found in the original bone, although not necessarily in their original quantities. In other embodiments of the present invention, blocks of cortical bone are demineralized, and the fibers are subsequently produced by crushing or shredding the demineralized blocks.

The demineralization process is carefully controlled via the concentration of acid and duration of soak time in order to enhance the mechanical properties of the fibers while retaining the osteoinductive components that are exposed by the dilute acid reagents. Following demineralization, the tissue goes through a pH restoration process where the residual acid is neutralized by buffering reagents thereby returning the tissue to near physiological pH of between 6-8 pH. Subsequently, the demineralized cortical bone fibers may be stored in a wet state or dried using lyophilization or other drying techniques. The DCBF may be stored at various temperatures including but not limited to ambient room temperature (e.g., at about 23° C.), refrigerated (e.g., at about 4° C.), frozen (e.g., at about −20° C.), or cryogenically preserved (e.g., at about −196° C.) using controlled rate or uncontrolled rate freezing.

The DCBF may be placed into an implant forming container, such as a jar or a mold, and formed into a variety of shapes including, but not limited to, thin sheets, cubes, discs and strips. More intricate geometries may also be formed including, but not limited to, curves, cutouts, compartments and patterning which can be determined by the shape of the implant forming container. DCBF stored in a wet state may be placed, for example, into molds directly, whereas dried DCBF will need to be rehydrated prior to being placed in molds. For example, when dried DCBF are used they are first disbursed into a liquid carrier to form a solution and then agitated to ensure even distribution of the DCBF in the solution in the mold. As also discussed hereinbelow, the liquid carrier used to form the solution may be, for example without limitation, water, aqueous saline solution, Sorensen's buffer, or phosphate buffered saline solution. In some embodiments, excess liquid from the wet or rehydrated tissue may be separated from the DCBF, drained and removed from the mold. In some embodiments, additional liquids (e.g. water, buffer, or saline) may be added to the tissue before and during the molding process. The liquids added to tissue before and during the molding process could optionally contain therapeutic factors, cytokines, growth factors, pharmaceuticals, antibiotics, free-radical scavengers, sugars, vitamins including, but not limited to, riboflavin and ascorbic acid, surfactants, DMEM medium, human or animal serum, or other additives. The addition or removal of liquid from the tissue also allows the density of the final implant to be controlled and production of an implant of uniform density. The mold may be composed of a single or multiple types of materials, including but not limited to metals, glass, plastics, silicone, Teflon®, and ceramics. In an embodiment, the vessel or package in which the demineralized cortical bone fibers are stored serves as the mold.

In an embodiment, the mold is micro-porous or meshed with pore sizes ranging up to 5 mm. In an embodiment, the mold includes a non-uniform material. In an embodiment, the mold has varying pore sizes or mesh sizes, with the pores or meshes having different sizes at different locations in the mold. In an embodiment, the mold may include a layer of material placed on the top of the DCBF, the layer being of the same material used elsewhere in the mold or of a different material. In embodiments, the layer is solid, porous, or meshed, or has another geometry appropriate to the intended use of the mold and implant to be produced therefrom.

In an embodiment, DCBF are in the form of a mass of DCBF, which are then used to prepare implants that may be used as bone void filler or bone graft extender in bony voids and gaps which have been surgically created or caused by traumatic injury to the bone. Implants and grafts, as used herein, refer to tissues, organs or components thereof that are transplanted from a donor to a recipient and include those transplanted between individuals of the same species (“allograft”), those donated and transplanted into the same individual (“autograft”), and those transplanted between individuals of different species (“xenograft”). Such implants may be used as a standalone treatment device or be applied in combination with one or more of a variety of bioactive osteogenic materials or cells that facilitate the reconstruction and healing of bone. Such implants may include particles of cortical, cancellous, or corticocancellous bone. Such particles may be partially demineralized, demineralized, fully demineralized, or may have most or all of their original mineral or calcium content.

In an embodiment, the DCBF are pre-hydrated in an aqueous buffer, or combined with a carrier, such as, but not necessarily limited to, the following: an isotonic solution; a sodium chloride solution at a concentration of about 0.1% to about 1%, more particularly, about 0.9%; a lactated Ringer's solution, with or without D5LR; phosphate buffered saline (“PBS”); platelet rich plasma (PRP); glycerin; lecithin; alginate; hyaluronic acid (HA); a derivative of HA; or sodium hyaluronate; or other suitable carriers known in the art. The term “carrier” as used herein refers to a pharmaceutically acceptable inert agent or vehicle for delivering one or more active agents to a subject, and often is referred to as “excipient.” The carrier must be of sufficiently high purity and of sufficiently low toxicity to render it suitable for administration to the subject being treated. The carrier may also comprise “biological components” added to the carrier, such as, but not limited to, DNA, RNA, short hairpin RNA (shRNA), small interfering RNA (siRNA), micro RNA (mRNA), polysaccharides, peptides, matrix proteins, glycosaminoglycans (e.g, hyaluronic acid), viral vectors, and liposomes. The carrier further should maintain the stability and bioavailability of an active agent added to the carrier.

In an embodiment, a mass of DCBF fibers (e.g., an implant) are provided to a surgeon, who can then add one or more of a carrier, bone marrow, blood, non-demineralized bone chips, etc., and then mold or reshape the mass into a preferred configuration according to anatomical or surgical needs in the operating room. The final form should be cohesive, moldable, and provide some resistance to irrigation when in the defect site, and leave minimal residue on the gloves of those handling it. When the mass is thus prepared, the surgeon can place it in a bone defect site, a site with two adjacent bone defects, or any non-bony defect where it is desired to form new bone or repair bone.

### II. CHEMICAL AND SURFACE TREATMENT OF DEMINERALIZED CORTICAL BONE FIBERS

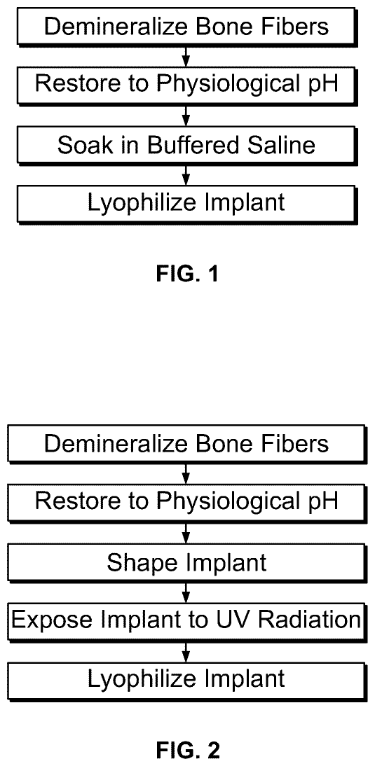

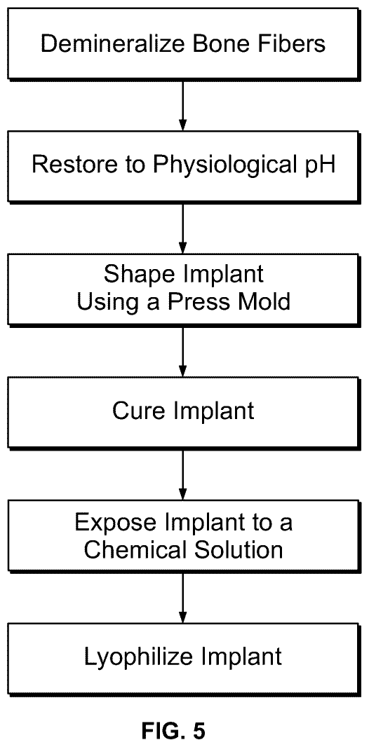

In an embodiment of the present invention, DCBF are prepared as described in Section I, above, and subjected to treatment with one or more chemical solutions to improve the wettability of the individual fibers and of the fibrous mass. The increased wettability can be obtained by changing the surface charge of the DCBF or changing the surface morphology and/or micro-geometry of the DCBF. The fibers or fibrous mass may be treated with such chemical solutions immediately before the pH restoration step, after the pH restoration step, or before the fibers or fibrous mass are dried. In an embodiment, the fibers or fibrous mass may be dried, then rehydrated prior to treatment with the chemical solution. Furthermore, the DCBF may be treated with such chemical solutions after formation of the implant and before any final drying or lyophilizing step, where applicable. Simplified flow charts of representative chemical treatment processes are shown in FIGS. 1 and 5.

The chemical treatment is performed by contacting the DCBF with one or more chemical solutions selected to improve the wettability of dried or lyophilized DCBF. In an embodiment, the DCBF are soaked in the chemical solution for a period of time from about 6 hours to about 48 hours, for example from about 12 hours to about 36 hours, for example from about 20 hours to about 28 hours. In an embodiment, the soak is a static soak. In an embodiment, the DCBF are agitated during the soak.

In an embodiment, the chemical solution is isotonic with blood. In an embodiment, the chemical solution includes a dissolved salt. In an embodiment, the chemical solution is a physiologically-balanced solution that includes a salt. In an embodiment, the chemical solution is a saline solution. In an embodiment, the solute in the chemical solution consists of sodium chloride (e.g., a 1 M NaCl solution). In an embodiment, the chemical solution is Ringer's solution. In an embodiment, the chemical solution is a buffer solution containing a buffering salt. In an embodiment, the chemical solution includes a phosphate salt. In an embodiment, the buffer solution is a standard buffering solution containing a buffering salt. In an embodiment, the buffer solution is a standard phosphate buffered solution (e.g., PBS). In an embodiment, the chemical solution is Sorenson's Buffer. In an embodiment, the chemical solution is Hanks Buffered Salt Solution. In an embodiment, the chemical solution is a HEPES-buffered solution.

In an embodiment, the chemical solution includes a biologically-compatible polar organic compound. In an embodiment, the chemical solution includes an alcohol. In an embodiment, the chemical solution includes ethanol. In an embodiment, the chemical solution includes a polyol. In an embodiment, the chemical solution includes a glycol. In an embodiment, the chemical solution includes glycerol. In an embodiment, the chemical solution includes polyethylene glycol. In an embodiment, the chemical solution includes a sugar. In an embodiment, the chemical solution includes dextrose. In an embodiment, the chemical solution includes mannitol-D. In an embodiment, the chemical solution includes sodium ascorbate. In an embodiment, the chemical solution includes one or more of a ketone, an aldehyde, an organic acid, or another biocompatible polar organic compound. In an embodiment, the chemical solution includes an additive to inhibit proteolytic activity of proteinases (e.g., matrix metalloproteinases, “MMP”). In an embodiment, the additive is chlorhexidine gluconate. In an embodiment, the additive is galardin. In an embodiment, the chemical solution includes a combination of one or more biologically-compatible polar organic compounds and one or more dissolved salts. In an embodiment, the chemical solution is a non-aqueous solution. In an embodiment, a polar organic liquid is used in place of the chemical solution.

In an embodiment, the chemical solution includes a biologically-compatible polar organic compound and/or a dissolved salt, and an additive. In an embodiment, the additive is a therapeutic agent for administration to a mammal. In an embodiment, the additive is a cytokine. In an embodiment, the additive is a pharmaceutical. In an embodiment, the additive is an antibiotic. In an embodiment, the additive is a nutrient. In an embodiment, the additive is a trace element. In an embodiment, the additive is a free-radical scavenger. In an embodiment, the additive is a growth factor. In an embodiment, the additive is a biologically-active compound.

In an embodiment, the ratio of DCBF to the chemical solution is in a range of about 1:10 g/ml to about 1:1 g/ml. In an embodiment, the ratio of the DCBF to the chemical solution are selected to provide a desired fiber density and fractional void volume in the dried implant. In such an embodiment, lower ratios of DCBF to chemical solution result in less dense implants with higher void volumes.

In some embodiments, the implants produced by the methods described and contemplated herein have uniform density. An implant may be tested for uniform density by various methods. One suitable method, for example without limitation, for determining whether an implant has uniform density is to measure the overall density of the implant, then divide or cut the implant into at least three portions and measure the density of each portion, to produce at least four measured density values for that single implant. The average density for that implant is calculated by dividing the sum of all densities (whole implant and all pieces) by the total number of pieces plus 1 (for the whole implant density). Next, the percent relative standard deviation (% RSD) for that implant is determined as a percentage by first determining the standard deviation of all the measured density values using conventional statistical analysis methods, and then dividing that standard deviation by the average density and multiplying by 100. As the term “uniform density” is used herein, an implant is considered to have uniform density when the % RSD is less than about 30%, such as less than about 25%, or less than about 20%, or less than about 15%, or less than 10%. Example 26 provides an example of such calculations.

Following treatment with the chemical solution, the treated DCBF are dried. In an embodiment, the treated DCBF are dried by air drying. In an embodiment, the treated DCBF are dried by vacuum filtration. In an embodiment, the treated DCBF are dried by heat-drying. In an embodiment, the treated DCBF are dried by solvent-drying. In an embodiment, the treated DCBF have a residual moisture content of less than 80% after drying. In an embodiment, the treated DCBF have a residual moisture content in a range of about 60% to about 80% after drying.

In an embodiment, the treated DCBF are dried by lyophilization. In an embodiment, the treated DCBF are frozen before being lyophilized. In an embodiment, the treated DCBF are refrigerated before being lyophilized. In an embodiment, the treated DCBF are staged at room temperature before being lyophilized. In an embodiment, the treated DCBF are dried to a residual moisture content of less than 80% before being lyophilized. In an embodiment, a quantity of a chemical solution is added to the dried DCBF, and the solvent is removed from the DCBF fibers by lyophilization. In an embodiment, the ratio of treated DCBF to chemical solution is in a range of about 1:0.8 (g/ml) to about 1:10 (g/ml) before lyophilization. In an embodiment, the treated DCBF have a residual moisture content of less than 6% after lyophilization.

In an embodiment, wet-treated DCBF (i.e., DCBF treated with a chemical solution) are placed in an implant forming container, such as a mold, prior to lyophilization, such that the lyophilized DCBF mass takes the shape of the mold. In an embodiment, wet treated DCBF are placed in a jar or other container, then lyophilized. The final tissue form, or implant comprising treated dried DCBF, may then be provided to medical personnel for use as discussed in Section I, above.

As shown FIG. 5, in another embodiment, following treatment with a chemical solution and prior to drying or lyophilizing, the treated DCBF may be subjected to a curing step which involves warming the treated DCBF for a period of time. For example, curing may be accomplished, without limitation, by warming the treated DCBF using ambient air, warm air, radiant heat, or energy such as UV light or microwaves. In such an embodiment, the treated DCBF may be warmed to a temperature of from about 20° C. to about 50° C., such as from about 25° C. to about 45° C., or from about 30° C. to about 45° C., or from about 35° C. to about 45° C. In such an embodiment, the treated DCBF may be warmed for a period of time of from about 30 minutes to about 24 hours, such as from about 4 hours to about 20 hours, or from about 4 hours to about 16 hours, or from about 6 hours to about 12 hours. Without intending to be limited by theory, it is believed that performing a warming step as described above produces an implant comprising treated DCBF that retains its shape after rehydration prior to use.

In an embodiment of the present invention, lyophilized DCBF treated using the methods described above are rehydrated prior to use. In an embodiment, lyophilized DCBR treated according to the methods described above are rehydrated prior to being packaged. In embodiments of such rehydration, the lyophilized DCBF are mixed with PBS, with or without other of the substances described above with respect to chemical solutions. In an embodiment, ratio of DCBF/PBS is selected to generate a cohesive, moldable composition that includes completely hydrated DCBF. In an embodiment, the mixture is in a range of about 20:80 DCBF/PBS (g/ml) to about 34/66 DCBF/PBS (g/ml).

In an embodiment of the present invention, the surface roughness of the DCBF is modified using a surface modification technique known in the art or to be discovered. Known suitable techniques include, without limitation, overcoating, surface gradient modification, surface-active bulk additives, surface chemical reactions, etching, roughening, conversion coatings, ion beam implantation, Langmuir-Blodgett deposition, laser roughening, parylene coatings, photografting, radiation grafting, radiofrequency glow discharge plasma deposition, radiofrequency glow discharge treatment, self-assembled monolayers, silanization, surface-modifying additives, and other means of modifying surfaces of fibers. In an embodiment, one or more of the aforesaid techniques creates surface features on the micron scale, sub-micron scale, nano-scale, or other scales.

In an embodiment of the present invention, the wettability of an implant comprising modified or non-modified DCBF can be measured using standard methods for assessing surface tension, including but not limited to static and dynamic contact angle measurement techniques. Suitable contact angle measurement techniques include, but are not limited to, optical tensiometry, force tensiometry, Wilhelmy plate methods, sessile drop methods, captive air bubble methods, capillary air methods, the du Nouy ring method, or other measurement techniques for determining contact angles of liquid substances. In certain embodiments, DCBF implants prepared according to methods of the present invention may have at least one surface where the contact angle is less than 90 degrees, or less than 60 degrees, or less than 45 degrees. Another suitable method for measuring the wettability of an implant is, for example without limitation, by observing the rate at which a DCBF implant absorbs an amount of liquid. In an embodiment, the amount of liquid is a measured volume deposited on a surface of the implant and the measured value is known as wettability time. Implants produced according to the methods described and contemplated herein have a wettability time of less than about 5 minutes, such as less than about 4 minutes, or less than about 3 minutes, or less than about 2 minutes, or less than about 1 minute. Still another suitable method for measuring the wettability of an implant is, for example without limitation, submerging the implant in an excess amount of liquid and measuring the time required for the implant to absorb enough of the liquid to completely submerge the implant and the measured value is known as complete rehydration time. Implants produced according to the methods described and contemplated herein have a complete rehydration time of less than about 30 minutes, such as less than about 20 minutes, or less than about 15 minutes, or less than about 10 minutes, or less than about 5 minutes.

### III. ENERGETIC, PHYSICAL, AND CHEMICAL CROSS-LINKING OF DEMINERALIZED CORTICAL BONE FIBERS

In an embodiment, the present invention includes an implant that is comprised of DCBF that have been either fully or partially cross-linked using energetic sources. Suitable energetic sources include ultraviolet (UV) radiation, ozone, plasma, (e.g., RF plasma), coronal discharge, or other means that provide the energy needed to form cross-links between proteins. Suitable plasma media include, but are not limited to, air plasma, oxygen plasma, and ammonium plasma. In an embodiment, energetic cross-linking binds proteins such as albumin or other blood adsorption proteins to the DCBF, otherwise affects the adsorption of the proteins to the DCBF, before lyophilization to increase the wettability of the DCBF implant. The wettability of the energetically cross-linked DCBF implants is measured using the same techniques described in Section II with regard to the chemical and surface treatment of DCBF.

Cross-linking imparts a variety of unique properties to the DCBF implant that a non-cross-linked implant would otherwise not possess. Such properties include increased wettability, shape retention under compression, and resistance to fiber washout. A simplified flow chart of a representative energetic cross-linking treatment process is shown in FIG. 2.

In an embodiment of the present invention, DCBF are cross-linked by exposing a mass of DCBF to UV radiation. In an embodiment, wet DCBF are placed into a mold and formed into one of a variety of possibly desirable shapes. Such shapes include, but are not limited to, thin sheets, cubes, discs and strips. More intricate geometries may also be formed including, but not limited to, curves, cutouts, compartments and patterned shapes. In an embodiment, the mass is shaped to approximate a surface of an intact or damaged bone, such as to line a hip socket or the interior of a bone void.

In embodiments of the present invention, suitable molds may be composed of single or multiple types of material or combinations of materials. Such materials include, but are not limited to metals, glasses, plastics and ceramics. Suitable materials may either block UV radiation completely, partially transmit, or fully transmit UV radiation, allowing all or selected portions of the implant to be exposed to UV radiation. While most materials exhibit poor transmission of UV radiation, certain materials such as fused quartz or silica glass and plastics including, but not limited to, optical grade polystyrene and specialized PMMA acrylic (Plexiglas G-UVT, Solarcryl SUVT, Acrylite OP-4) allow for near full transmission of certain wavelengths of UV radiation. After molding the sample into its final shape, the implant may be left in the mold or removed from the mold before undergoing UV cross-linking. The implant may be lyophilized within or without the mold before undergoing UV cross-linking, or lyophilized and rehydrated again prior to UV cross-linking. The implant may also be further masked using materials that completely block or are partially transmissible to UV radiation to further control cross-linking in certain regions of the implant.

In embodiments of the present invention, the mold is a composite of various materials selected to provide variations in the degree of cross-linking across the implant. In an exemplary embodiment, the implant is formed with a cavity to receive an osteoinductive substances or other therapeutic material. In such an embodiment, it may be desirable that the bottom of the implant, opposite the cavity, may be more densely cross-linked to provide increased structural stability to the implant. In other embodiments, variations in cross-linking density may be used to allow certain sections of the implant to be remodeled at different rates than other sections during the bone remodeling process.

In an embodiment of the present invention, UV surface cross-linking is performed by placing the implant in a UV containment chamber and exposing the implant to UV radiation. The UV radiation alters the collagen molecules within the implant, resulting in additional bonds being formed between adjacent collagen molecules. This process of photopolymerization of collagen is believed to occur due to the generation of free radicals via photooxidation of sensitive amino acid residues by UV radiation. The free radicals generated allow the formation of covalent cross-links between the collagen polypeptides, resulting in stronger and stiffer collagen fibers. Aromatic amino acid residues are the predominant sites of free radical formation. Other amino acid residues may be the site of free radical generation under more energetic conditions. Further, the rate at which cross-linkages are formed may be increased by adding biologically-compatible free radical initiators to the DCBF mass. Riboflavin is an example of such an initiator. Other initiators may include other compounds with aromatic structures, or may include sugars.

The amount of liquid in the implant affects the rate and degree of cross-linking. Without being bound by theory, it is believed that the presence of liquid provides a medium for transport of free radicals between collagen fibers. While it is possible to cross-link dried or lyophilized fibers, the embodiments of cross-linking methods according to the present invention are most effective when used with rehydrated fibers. However, excess water may be added to the DCBF implant before cross-linking to swell the implant, thus increasing its porosity, and the exposure time increased, if necessary to achieve the desired amount of cross-linkage.

The rate and depth at which cross-linkages are formed may be controlled by altering the power of the UV radiation source, changing the distance of the implant from the UV radiation source, shifting the wavelength of the UV radiation, varying the exposure time, and by fully or partially blocking UV radiation transmission to certain areas of the implant. Multiple UV radiation sources may be used with a combined power rating ranging from a few watts to a few kilowatts. In high power or energy dense cross-linking implementations of the present invention, the UV containment chamber and implant may be cooled to temperatures ranging from physiological (e.g., about 37° C.) to freezing (e.g., about −80° C.) during the cross-linking process using any of a variety of cooling techniques to prevent heat-related degradation of the implant. Suitable cooling techniques include but are not limited to refrigerant-based cooling, active air cooling, thermoelectric devices, evaporative cooling, and phase-change cooling (e.g., the use of dry ice). The implant may also be placed under UV radiation for multiple short exposures instead of a single long exposure to reduce the amount of heat generated in the tissue. In some embodiments that involve UV cross-linking, it may be beneficial to heat the implant to a temperature that is higher than physiological temperatures (e.g., the implant may be heated to a temperature in a range of from about 37° C. to about 70° C.). Heat may be applied to the implant by the UV bulbs or an additional heating element. The implant may be placed on a heating platform and/or heated by UV bulbs placed around the implant. The addition of heat greater than about 37° C. but less than about 70° C. for lengths of time of from about 10 minutes to about 24 hours increases the cohesiveness of the implant and helps prevent dispersion of the implant when rehydrated or submerged in a rehydrating liquid (e.g., water, saline, blood). In some embodiments, the use of heat to improve the cohesiveness of the implant may be used without the addition of UV exposure.

In embodiments of a method according to the present invention, the intensity or irradiance of the UV radiation at the surface of the implant may be varied by the power of the radiation source and/or the distance between the implant and the UV radiation source. Suitable energy densities for use in a method according to an embodiment of the present invention range from about 100 μW/cm² to about 5,000 mW/cm² at the surface of the implant. The wavelength of the UV radiation can be shifted between various regions of the UV spectrum including but not limited to longwave UVA (e.g., about 400 to about 315 nm), midrange UVB (e.g., about 315 to about 280 nm), and shortwave UVC (e.g., about 280 to about 100 nm). Shifting the wavelength changes the penetration properties of UV radiation into the implant, with longer wavelengths allowing increased UV penetration and greater depth of cross-linking. For example, in an embodiment of the present invention, exposure to UVA radiation is used to create cross-linking to a depth of about 1 mm, which creates a stiff shell at the surface of the implant. Shifting wavelengths also changes the character of the cross-links, which affects the degree to which properties such as mechanical strength, shape memory retention, and hydrophobicity are modified. Concurrent exposure to UV radiation at differing wavelengths may be used to vary the changes in properties across the implant. Wavelengths in the UVC spectrum also have the added benefit of being germicidal, and thus can be used to sterilize the surfaces of the implant while it undergoes cross-linking.

The length of time that the implant is exposed to the UV radiation source also affects the degree and effectiveness of cross-link formation. In cross-linking methods according to embodiments of the present invention, suitable exposure times are in a range of a few seconds to a few hours depending on the desired properties of the implant. In some embodiments, exposure times of up to 720 minutes may be used, although typical exposure times of about 10 minutes or less may be used (e.g., for commercial production of implants). In some embodiments, even shorter exposure times (e.g., exposure times of about 10 seconds to about 300 seconds) may be used where only a small degree of cross-linking is desired, or where the UV radiation is particularly intense. For many embodiments, the practical exposure times would be in a range of about 10 minutes to about 60 minutes.

After the cross-linking process is completed, the implant may be stored in a wet state or dried using lyophilization, air drying, or other drying methods. The implant may be stored at various temperatures including but not limited to ambient room temperature (e.g., at about 23° C., or up to about 30° C.), refrigerated (e.g., at about 4° C.), frozen (e.g., at about −20° C.), or at cryogenic temperatures (e.g., at about −196° C.) where frozen or cryogenic freezing is achieved using controlled rate and/or uncontrolled rate freezing. By changing the variables discussed above before and during the cross-linking process, a broad range of implants with varying properties may be produced.

In an embodiment of the present invention, the cross-linking process is performed in a containment chamber that allows optimal UV irradiation while shielding an operator from potentially harmful UV irradiation. During the cross-linking process, the implant may be placed on a flat surface, an uneven surface with ridges and peaks, or elevated on a platform or by other means that would allow UV radiation to reflect onto all sides of the implant, including its underside. The surface or platform that the implant rests on could also be made of multiple types of materials that block UV radiation completely, partially transmit UV radiation, or fully transmit UV radiation. The walls of the UV containment chamber may be lined or coated with a reflective material to allow the radiation to scatter within the UV containment chamber, allowing all surfaces of the implant to be exposed to UV radiation. UV radiation sources may also be mounted on multiple walls of the UV containment chamber to allow for better coverage of the implant during the cross-linking process. The orientation of the implant may also be changed during the UV cross-linking process either manually or automated by the UV containment chamber for a more uniform exposure of all surfaces.

Embodiments of the UV cross-linking method of the present invention include the aforesaid containment chambers, which may be specially designed to meet the needs of specific embodiments of the UV cross-linking method. Containment chambers according to embodiments of the present invention may also be designed for use with energetic sources other than UV radiation sources, such as ozone, plasma, (e.g., RF plasma), coronal discharge, or other means that provide the energy needed to form cross-links between proteins. In an embodiment, the containment chamber includes means for positioning and/or moving the implant. In an embodiment, the containment chamber includes one or more sources of UV radiation.

In an embodiment of the present invention, the distance of an implant from a UV radiation source may be changed during the irradiation process using manually or automatically operated device to provide optimal UV irradiation for different types of implants. In an embodiment, the device includes a manual or automated moving platform upon which the implants rest. Such platforms can move along x-, y-, and z-axes. In an embodiment, the device includes single or multiple UV radiation sources that can move along x-, y-, and z-axes. In an embodiment, the UV radiation source is one or more UV lamps in a movable lamp fixture. In an embodiment, the device includes a rotating drum. In an embodiment, the device includes a rotating platform. In an embodiment, the device includes an orbiting platform.

The effectiveness of the irradiation process may be affected by the temperature of the implant and/or UV radiation source. In an embodiment of the present invention, the containment chamber includes a temperature control system for regulating the temperature of the implant during irradiation by heating or cooling the implant. In an embodiment of the present invention, the containment chamber includes a temperature control system for heating or cooling the radiation source. In an embodiment, the interior of the UV containment chamber is ventilated and/or cooled using one or more input and output ports to control heating of the implant during the UV irradiation process. In an embodiment, such ventilation and/or cooling is controlled by a controller that is operated manually or automatically in response to temperature measurements made at the implant or elsewhere in the interior of the containment chamber.

In an embodiment of the present invention, the UV radiation source includes one or more of a fluorescent lamp, a gas discharge lamp, a high-intensity discharge lamp, an electroluminescent lamp, a light-emitting diode, a laser, an incandescent lamp, an electron-stimulated lamp, and other devices that emit UV radiation at intensities suitable for cross-linking DCBF.

In an embodiment, a UV radiation controller is integrated in the containment chamber. The UV radiation controller includes one or more of means for opening and/or closing a shutter, means for turning one or more UV radiation sources on and/or off, means for controlling the brightness of the UV radiation source, and other means for controlling the intensity and/or duration of the irradiation of the implant. In an embodiment, a controller is provided, the controller having circuitry for controlling one or more of the aforesaid means. In an embodiment, the controller includes a computer. In an embodiment, the computer is programmable by an operator.

In an embodiment, the containment chamber includes one or more sensors to sense the intensity of UV radiation emitted by the UV radiation sources and/or the intensity of UV radiation at the surface of the implant. In an embodiment, a controller is provided, the controller having circuitry for controlling the intensity of the UV radiation source. In an embodiment, the controller controls the intensity of the UV radiation source in response to output from the one or more sensors. In an embodiment, the controller includes a computer. In an embodiment, the computer is programmable by an operator such that the UV radiation source provides UV radiation of a specified intensity and/or range or wavelengths. In an embodiment, the computer is programmable by an operator such that the UV radiation source provides a total irradiation energy to the implant.

In an embodiment, the UV containment chamber is designed to be used in one or both of a sterile and a non-sterile environment. In an embodiment where the environment is non-sterile, the implant is contained in a sterile interior of a separate UV-transmissive chamber that is placed in the UV containment chamber such that radiation from the UV radiation source is transmitted through the UV-transmissive chamber to the implant. In an embodiment, the interior of a UV containment chamber is maintained as a sterile environment by sealing the UV radiation source and controller circuitry in a separate compartment. In such an embodiment, the sealed compartment is UV transmissive such that UV radiation from the UV radiation source is transmitted from the sealed compartment into the interior of the UV containment chamber.

UV cross-linking of DCBF provides an implant with properties that an otherwise non-cross-linked implant would not possess. The current lyophilized formulations of demineralized cortical fibers have a few shortcomings that can be address by UV cross-linking. One such shortcoming is the initial resistance to rehydration of a lyophilized DCBF implant. When the implant has been lyophilized, the residual moisture level is typically no more than 6% w/w and this lack of moisture causes the implant to exhibit hydrophobic characteristics. When a liquid such as water, saline, or blood is applied to the surface of the implant, the liquid sits on the surface and is not immediately absorbed. Once the initial amount of liquid becomes absorbed into the implant, the rehydrated surface exhibits hydrophilic characteristics and any additional liquid added is immediately absorbed into the implant. Another shortcoming is the lack of mechanical strength and structural rigidity of a lyophilized DCBF implant after rehydration. In the lyophilized state, the implant holds its shape and is rather stiff, however, after the implant has been rehydrated, the implant becomes soft, the DCBF start to swell, and the implant cannot be handled without permanently losing its shape. In certain situations, it is preferable for the implant to retain its shape while also being compliant and flexible even after being saturated with liquid.

UV cross-linking allows the hydrophilicity and mechanical properties of a DCBF implant to be modified quickly and efficiently compared to other methods known in the art. However, an embodiment of the present invention include physical cross-linking by techniques such as those including dehydrothermal treatment (DHT). An embodiment of the present invention includes chemical cross-linking of DCBF by one or more known methods, or by a chemical cross-linking method yet to be discovered.

Known chemical cross-linking techniques include, but are not limited to, the use of glutaraldehyde, carbodiimide (e.g., 1-ethyl-3-(3-dimethylaminopropyl) carbodiimide, also known as EDC), EDC with NHS (i.e., N-hydroxysuccinamide), genipin, catechin, succinic acid, and tannic acid. While some chemical cross-linkers have been used in the past on various types of materials, including allograft tissue, chemical cross-linking can be a complicated and lengthy process, and is potentially hazardous to the patient if the residual chemicals are not completely removed. Natural chemical cross-linkers, such as genipin and catechin, are less cytotoxic than synthetic cross-linkers, but may also have disadvantages in some applications. In the case of genepin, the tissue is stained a dark blue as a result of the cross-linking process, and the stain is difficult to remove. Chemical cross-linking is also difficult to control and is more easily applied to the entire bulk of the implant rather than to specific areas or surfaces. Too much cross-linking of the implant may also impart properties that are unfavorable. One of the advantages of implants made from DCBF is that they are moldable and cohesive after rehydration. This property is diminished as the DCBF become more cross-linked, resulting in an implant that cannot be molded into a different shape or put together once it has been taken apart. Despite the aforesaid difficulties posed by chemical cross-linking techniques, their use in forming cross-linked implants, as well as the implants themselves, are useful embodiments of the present invention.

In contrast to the chemical cross-linking methods discussed above, cross-linking by UV radiation is easily controlled and can be implemented to prepare DCBF implants that have the advantages of both non-cross-linked and cross-linked DCBF, while eliminating the disadvantages of excessive stiffness and resistance to recombination of pieces of the implant. By using UV radiation to cross-link certain surfaces of the implant while leaving other areas uncross-linked, an implant is prepared that retains its shape after rehydration due to the increased stiffness of the cross-linked regions, while also retaining the moldable and cohesive properties of the uncross-linked regions. UV cross-linking also reduces the initial hydrophobicity encountered by the lyophilized demineralized cortical fibers allowing the implant to be rehydrated nearly instantaneously. Furthermore, UV cross-linking imparts some shape memory retention to the rehydrated implant. When an external force is applied, the cross-linked implant is temporarily deformed and some liquid is displaced. However, as soon as the force is removed, the cross-linked implant will return to its original shape and resorb the previously displaced liquid. Only when a sufficient amount of force is applied does the implant permanently deform and become moldable. Additionally, the increased rigidity of the cross-linked surfaces of the implant prevents the implant from breaking apart when an excess of liquid is applied, when the implant is irrigated, or when the implant is completely submerged in a liquid.

Embodiments of the cross-linking methods of the present invention can be used to produce hydrophilic and mechanically stable DCBF implants from fully demineralized, demineralized, or partially demineralized DCBF, but is most effective for cross-linking DCBF with calcium contents of less than 1% w/w. The UV cross-linking method of the present invention may be used with DCBF having thicknesses in a range of about 80 μm to about 150 μm, or at other thicknesses where the DCBF form a cohesive mass in the absence of cross-linkages. Further, embodiments of the energetic method of the present invention can be used to prepare DCBF implants in the presence of additives. Additives such as particles of non-demineralized cortical, cancellous, or corticocancellous bone, demineralized cortical, cancellous, or corticocancellous bone may be used as long as the implant contains sufficient DCBF to form a cohesive mass. Additives such as therapeutic factors, cytokines, growth factors, pharmaceuticals, antibiotics, free-radical scavengers, sugars, or other chemical or bioactive compounds will retain their effectiveness after exposure, since the energetic exposure, and thus cross-linking, occurs at and/or near the surfaces of the implant, and does not significantly affect the interior of the implant.

Although the exemplary embodiments of the energetic cross-linking process described herein discuss the use of UV radiation, one having ordinary skill in the art and possession of the present disclosure will recognize that other sources of energy may be used to cross-link protein-rich fibers. Besides UV radiation, suitable energetic sources include, but are not limited to, ozone, plasma, (e.g., RF plasma), coronal discharge, or other means that provide the energy needed to form cross-links between proteins. Suitable plasma media include, but are not limited to, air plasma, oxygen plasma, and ammonium plasma.

### IV. TREATMENT OF TISSUE TYPES OTHER THAN DEMINERALIZED BONE

Without being bound by theory, it is believed that the increased wettability and other effects observed in DCBF and masses of DCBF that have been treated as discussed herein result from interactions with the collagen and/or glycoproteins present in cortical demineralized bone matrix. Thus, one having ordinary skill in the art and possession of the present disclosure would reasonably expect that similar beneficial results may be obtained by applying such treatments to demineralized bone matrix from cancellous or corticocancellous bone. One having ordinary skill in the art and possession of the present disclosure would also reasonably expect that similar beneficial results may be obtained by applying such treatments to fibers or other particles of tissue types other than demineralized bone matrix. Such other tissue types may be derived from any suitable organ or other tissue source, whether autologous, allogeneic, or xenogeneic. Examples of suitable xenogeneic sources of tissues include, but are not necessarily limited to, warm-blooded vertebrates, including mammals, such mammalian sources including human, bovine, ovine, caprine, and porcine sources. Suitable tissue types may include, but are not necessarily limited to an adipose tissue, an amnion tissue, an artery tissue, a bone tissue, a cartilage tissue, a chorion tissue, a colon tissue, a dental tissue, a dermal tissue, a duodenal tissue, an endothelial tissue, an epithelial tissue, a fascial tissue, a gastrointestinal tissue, a growth plate tissue, an intervertebral disc tissue, an intestinal mucosal tissue, an intestinal serosal tissue, a ligament tissue, a liver tissue, a lung tissue, a mammary tissue, a meniscal tissue, a muscle tissue, a nerve tissue, an ovarian tissue, a parenchymal organ tissue, a pericardial tissue, a periosteal tissue, a peritoneal tissue, a placental tissue, a skin tissue, a spleen tissue, a stomach tissue, a synovial tissue, a tendon tissue, a testes tissue, an umbilical cord tissue, a urological tissue, a vascular tissue, a vein tissue, and a combination thereof. Other suitable tissue types may include, but are not necessarily limited to, submucosa, renal capsule membrane, dermal collagen, dura mater, serosa, or basement membrane layers, including liver basement membrane. Suitable submucosa materials for these purposes include, for instance, intestinal submucosa, including small intestinal submucosa, stomach submucosa, urinary bladder submucosa, and uterine submucosa. Source tissue (i.e., tissue incorporated into a final processed product, such as an implant) of the types disclosed above may be separated from other tissue types adjacent or connected to the source tissue, or the adjacent or connected tissue may remain with the source tissue and become incorporated in the implant. One or more source tissues may be included in the final processed product.

### V. EXAMPLES

The following examples are set forth so as to provide those of ordinary skill in the art with an exemplary disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g., amounts, temperatures, etc.) but some experimental errors and deviations should be accounted for.

### Example 1: Fabrication of Demineralized Cortical Bone Fibers

Human long bone is recovered aseptically from a deceased donor and stored at 4° C. until ready for processing. The bone is debrided to remove soft tissue elements and the shaft of the bone is cut into cross-sections. The cortical bone is then cleaned using detergents/surfactants to remove residual blood and lipids from the bone surface.

To create DCBF, the bone sections are first shaved across the shaft of the bone using a controlled advancement rate of a lathe bit having a width approximately equal to the desired length of the bone fibers. The shaft segment is secured in a vice with a sufficient portion of the shaft protruding such that the protruding portion may be shaved. On a milling machine, a straight flute end-mill is set up such that its axis is parallel with the axis of the shaft. Utilizing the required length of the of the broad edge of the lathe bit, fibers are shaved off of the shaft by running the end-mill back and forth along the shaft until substantially all of the bone has been shaved from the shaft. The resulting bone fibers are collected for demineralization.

The bone fibers are demineralized by agitating them in 0.6 N HCl for a sufficient period of time to remove the endogenous calcium minerals to a desired residual calcium content, after which the fibers are successively rinsed with water, soaked in water, soaked in a sodium phosphate dibasic buffer to achieve a physiological pH, rinsed in water, and soaked in water. The soaked fibers may then be dried, lyophilized, or left in a wet state for further processing.

### Example 2: Treatment of DCBF with PBS

DCBF are prepared as described in Example 1. After completion of the second water soak, the DCBF are decanted into a vessel, and PBS is added at a ratio in a range of about 1:3 DCBF/PBS (g/ml) to about 1:15 DCBF/PBS (g/ml). After 5 to 15 minutes of a static soak, the DCBF are decanted from the PBS, and air-dried. Additional PBS is added to the DCBF at a ratio in a range of about 1:1 DCBF/PBS (g/ml) to about 1:5 DCBF/PBS (g/ml) in a plastic jar, and the wet DCBF are lyophilized.

### Example 3: Preparation of Low-Density Pre-Formed Fiber Shapes Using DCBF and Saline

Low-density pre-formed fiber shapes are lyophilized DCBF which are suspended in liquid prior to lyophilization to provide a fluffy texture and a high void volume. They are hydrated by a surgeon in the operating room to form a putty-like substance for use as a bone void filler.

Low-density pre-formed fiber shapes were prepared using water or different ratios of 0.9% sodium chloride in water (“saline”, in particular 0.25× saline, 0.5× saline, 0.75× saline, and 1× saline) to examine the effect of salt concentration on hydration time and handling properties of the implants. The samples prepared with water were used as control samples; the samples prepared with saline solutions were examined as test samples.

Samples of air-dried DCBF prepared according to Example 1 were soaked in water or saline at selected concentrations at a ratio in a range of about 1:3 DCBF/liquid (g/ml) to about 1:15 DCBF/liquid (g/ml) for 5 to 15 minutes, after which they were air-dried on a vacuum sieve. The samples were then lyophilized. Some samples were lyophilized in open jars; others were lyophilized with a vented lid, the ventilation holes having been covered by a porous liner having a pore size of greater than 10 μm. The lyophilized samples were then tested for hydration time and handling properties.

Test samples prepared with PBS and lyophilized with a lid and porous liner hydrated more rapidly than the control samples prepared with water. There was no significant difference in the handling of any of the test samples in comparison to the control samples.

### Example 4: Preparation of Low-Density Pre-Formed Fiber Shapes Using DCBF and PBS

Samples of air-dried DCBF prepared according to Example 1 were soaked in PBS at a ratio in a range of about 1:1 DCBF/PBS (g/ml) to about 1:5 DCBF/PBS (g/ml) for 5 to 15 minutes. Sets of samples were prepared using PBS at concentrations of 0.5×, and 0.25× of a standard PBS, using water as the diluent. After the soak, the samples were air-dried on a vacuum sieve, then lyophilized in open jars. The samples were then hydrated with just enough saline to provide good handling properties, and tested for appearance and hydration.

Drops of saline deposited onto the top surface of a low-density pre-formed fiber shape prepared with 0.25×PBS were absorbed in less than one minute. Drops of saline deposited onto the top surface of a low-density pre-formed fiber shape prepared with 0.5×PBS were absorbed more quickly.

### Example 5: Preparation of Low-Density Pre-Formed Fiber Shapes Using Various Ratios of DCBF and PBS

Samples of wet DCBF were prepared according to Example 1 without the final drying step. Samples of various sizes were soaked in a standard PBS at a ratio in a range of about 1:2 DCBF/PBS (g/ml) to about 1:5 DCBF/PBS (g/ml) for 5 to 15 minutes. After the soak, the samples were air-dried on a vacuum sieve, deposited in open jars, frozen, then lyophilized. The lyophilized samples were then tested for appearance and hydration. All of the samples had a fluffy appearance.

Low-density pre-formed fiber shapes prepared as described above were hydrated with sheep's blood, and the rates of absorption were compared with those of fiber shapes that had been prepared at a lower DCBF/PBS ratio in a range of about 1:2 (g/ml) and 1:5 (g/ml). Fiber shapes prepared at the higher ratio absorbed the sheep's blood at much faster rates than had been observed for the fiber shapes prepared at the lower ratio. The absorption rate was fastest for fiber shapes prepared at the highest ratio.

### Example 6: Comparison of Fiber Shapes Lyophilized with Water and Fiber Shapes Lyophilized with PBS

Samples of wet DCBF were prepared according to Example 1 without the final drying step. Two portions of wet DCBF were subjected to a static soak in standard PBS at a ratio in a range of about 1:3 DCBF/PBS (g/ml) to about 1:15 DCBF/PBS (g/ml), and air-dried. PBS diluted to 0.5× was added to a first portion at a ratio in a range of about 1:1 DCBF/PBS (g/ml) to about 1:5 DCBF/PBS (g/ml), and the DCBF was lyophilized in a plastic jar. Water was added to the second portion at a ratio in a range of about 1:1 DCBF/PBS (g/ml) to about 1:5 DCBF/PBS (g/ml), and the DCBF was lyophilized in a plastic jar.

Equal amounts of sheep's blood were dropped onto the lyophilized first (PBS) and second (water) portions of DCBF. The blood was entirely absorbed by the first portion within less than one minute, at which time only about one-third (⅓) of the blood was absorbed by the second portion.

### Example 7: Preparation of a DCBF Implant Containing Mineralized Granules of Cortical or Cancellous Bone

Samples of wet DCBF are prepared according to Example 1 without the final drying step. Mineralized granules or chips of cortical or cancellous bone having sizes in a range of about 200 μm to about 5 mm are prepared by milling or cutting of bone tissue which has been cleaned of any soft tissue adhering to the bone and treated with detergents/surfactants to remove blood and lipids. Following separate air-drying steps on individual vacuum sieves, mineralized cortical or cancellous granules/chips and DCBF are mixed in standard PBS at a ratio in a range of about 1:3 DCBF/PBS (g/ml) to about 1:15 DCBF/PBS (g/ml). The ratio of cortical or cancellous granules/chips to DCBF is in a range of about 1:0.1 to about 0.1:1 (g/g, based on air-dried weight), depending on the properties desired for the implant.

After mixing to obtain an approximately homogenous mixture, the resulting tissue mixture is air-dried on a vacuum sieve and deposited in jars which are subsequently filled with a volume of 0.5×PBS to re-suspend the tissue in liquid. The jars are sealed using lids with openings covered by porous liners, then frozen and lyophilized. Alternatively, after mixing and air-drying, the semi-wet tissue is placed into molds and lyophilized.

The lyophilized tissue is readily rehydrated with blood or saline and yields a moldable mass of bone tissue in which the cortical fibers provide cohesiveness and depending on their density within the tissue mass, the cortical/cancellous granules provide the implant with properties of radiopacity and/or resistance to compression.

### Example 8: Preparation of a DCBF Implant Having a Stiff Shell

Wet DCBF prepared as in Example 1 was placed into a rectangular mold and shaped into an implant having dimensions of approximately 10 cm×2.5 cm×7 mm. The fiber implant was removed from the mold and placed in a UV containment chamber where it was exposed to 315-400 nm UVA radiation for a period of about 30 minutes at an intensity in a range of about 4,000 μwatts/cm² to 20,000 μwatts/cm². The orientation of the implant was changed within the UV chamber during the irradiation process to expose all surfaces of the implant evenly to UV radiation, creating a stiff shell on all surfaces of the implant. The implant was then lyophilized for storage, and rehydrated prior to implantation.

### Example 9: Preparation of a DCBF Implant Having Compartments

Wet DCBF prepared as in Example 1 is placed into a rectangular mold having silicone inserts to form two large compartments on one surface of the implant. The resulting implant has dimensions of approximately 10 cm×2.5 cm×1.2 mm. The implant is removed from the mold with the silicone inserts in place. The implant is placed in a UV containment chamber where the exposed surfaces are exposed to both 100-280 nm UVC radiation and 315-400 nm UVA radiation. The longer UVA wavelength penetrates deeper into the surfaces of the implant, which imparts additional stiffness allowing the implant to retain its shape when rehydrated and loaded with additional materials in the compartments whereas the shorter UVC wavelength sterilizes the surfaces of the implant. The silicone inserts block the UVC and UVA radiation from reaching the interior of the cavities so that cross-linking does not occur at those surfaces. The resulting “boat” configuration implant has two open compartments that allow the user to add other materials such as bone marrow aspirate and cancellous chips, or other additives such as those discussed in Section III of the present disclosure. The interior surfaces of the compartments are not cross-linked, so that a user can mix the additives (e.g., the bone marrow aspirate and cancellous chips) into the non-cross-linked DCBF. After mixing, the user can pick up the implant in a single piece and fold it so as to close the compartments such that the additives are enclosed within the implant.

### Example 10: Preparation of a Thin DCBF Implant Having a Cross-Linked Interior

Wet DCBF prepared according to Example 1 are placed in a shallow rectangular mold to produce a thin strip-like implant with dimensions of approximately 10 cm×2.5 cm×2 mm. The implant is removed from the mold, and placed in a UV containment chamber where the exposed surfaces are exposed to 315-400 nm UVA radiation. The implant is thin enough that penetration of the UV radiation cross-links the majority of the DCBF in the interior of the implant. The resulting cross-linked implant is a porous and flexible strip that is also strong enough to be placed in areas of the body that are subject to mechanical loads that would disrupt implants having only a cross-linked shell.

### Example 11: Preparation of a Low-Density DCBF Implant

Wet DCBF prepared according to Example 1 are placed in a square mold to produce cube-shaped implants having dimensions ranging from about 5 mm to about 20 mm. Excess water is added to the mold to produce implants that are highly porous. The implants are then lyophilized and rehydrated carefully as to not disturb the porous structure. The implants are then placed into a UV containment chamber where the exterior surfaces are exposed to 315-400 nm UVA radiation. The implant is then lyophilized again. The resulting low density implant is highly porous yet is able to absorb liquids without swelling or deforming permanently.

### Example 12: Comparison of a Cross-Linked Implant with Non-Crosslinked Implant

Lyophilized DCBF prepared as in Example 1 was rehydrated and separated into portions. Each portion was placed into a customized cylindrical mold to produce puck-shaped implants. A first group of implants were irradiated with UVA radiation at an intensity in a range of about 4,000 μwatts/cm² to 20,000 μwatts/cm². The top and bottom of the first implant were irradiated for 15 minutes each, for a total exposure time of 30 minutes, for a total energy exposure in a range of about 180 Joules to about 900 Joules. The second group of implants were not irradiated, and served as comparison samples. The implants were then lyophilized. The implants had final dimensions of about 13 mm height and 29 mm diameter.

The implants were compared as follows:
1. Implants from the irradiated and non-irradiated groups were immersed in water or in saline solution. The implants from both groups remained intact. The implants from the irradiated group did not swell by any significant amount, but the implants from the non-irradiated group swelled by a considerable amount.
2. Implants from the irradiated and non-irradiated groups were compressed to a fraction of their initial size. Implants from the irradiated group showed better shape memory retention than implants from the non-irradiated group.
3. Rehydration of the lyophilized implants from both groups showed that the irradiated implants absorbed liquids more rapidly than the non-irradiated implants, and retained their shape better after rehydration even after being compressed significantly.
4. A moldability test showed that the irradiated implants became permanently deformed when sufficient pressure was applied to break the outer shell formed by cross-linking. The irradiated implants could then be molded into a variety of shapes.

### Example 13: Preparation of an Implant from Cartilage Fibers

Lyophilized fibers of articular cartilage, obtained by grating larger cartilage pieces, are rehydrated and placed in a cylindrical mold to produce plug-shaped implants having diameters in a range of about 5 mm to about 40 mm and lengths in a range of about 5 mm-to about 20 mm. The implants are removed from the molds, and placed in a UV containment chamber where the surfaces of the plugs are exposed to 315-400 nm UVA. The implant is then lyophilized and rehydrated. The resulting implant is compressible, but retains its shape when subjected to cyclical loading. The implant also stays in one piece and does not disperse when subjected to a load, during irrigation, or when placed in an aqueous environment.

### Example 14: Preparation of an Implant from Soft Tissue

Lyophilized tissues (e.g., fibers, flakes or powder) derived from placental (amnion, chorion, umbilical cord) or dermal tissue were rehydrated and compressed into thin sheets. The sheets were then trimmed or otherwise formed into a variety of shapes of varying sizes. The sheets were placed in a UV containment chamber where the surfaces of the sheets were exposed to 315-400 nm UVA radiation for about 20 minutes at a radiation intensity of about 20,000 μwatts/cm². The implant was then lyophilized and rehydrated prior to implantation. The resulting implant was a very thin sheet that retained its shape when flexed, perforated, irrigated, and placed in an aqueous environment.

### Example 15: Preparation of a DCBF Implant by Chemical Cross-Linking

Wet DCBF prepared as in Example 1 are placed into a rectangular mold and shaped into an implant having dimensions of approximately 10 cm×2.5 cm×7 mm. The fiber implant is removed from the mold and placed in a sealed vapor chamber. A solution of glutaraldehyde is heated within the chamber to generate glutaraldehyde vapors which penetrate and cross-link DCBF throughout the entire implant. The implant is exposed to the vapors for a set amount of time in a range of about 5 minutes to about 24 hours). After cross-linking, the residual unreacted glutaraldehyde and any unbound cross-linking byproducts are rinsed out of the implant using water, solutions of neutralization salts, and/or buffer solutions. The implant is then lyophilized for storage, and rehydrated prior to implantation.

### Example 16: Preparation of a DCBF Implant by Natural Cross-Linking

Wet DCBF prepared as in Example 1 are placed into a rectangular mold and shaped into an implant having dimensions of approximately 10 cm×2.5 cm×7 mm. A mesh is placed over the open surface of the mold to allow liquids access to the tissue while preventing the tissue from escaping the mold. The mold is submerged in a solution of genipin for a set amount of time in a range of about 5 minutes to about 24 hours. The genipin solution may remain static or can be stirred to increase the rate of cross-linking throughout the implant. After cross-linking, the residual genipin is rinsed out of the implant using water. Other rinses including detergents, salts, and/or buffers may be used to reduce residual staining of the tissue that occurs during the genipin cross-linking process. The implant is then lyophilized for storage, and rehydrated prior to implantation.

### Example 17: Preparation of a Hydrated DCBF Implant

Three grams of lyophilized DCBF was weighed out in a jar and between about 5 to about 7.5 ml of PBS was added to the DCBF. The two components were mixed, capped and let stand for more than 15 minutes at room temperature to ensure full homogenous hydration. The equilibrated mixture was packed into a plastic syringe, which was then capped and sealed in a foil pouch to prevent moisture loss during long-term storage. The mixture was extruded from the syringe into a pan and examined. The mixture was observed to have a slight off-white color, and to have a smooth consistency that held together when manually manipulated.

### Example 18: Preparation of a Shaped DCBF Implant with Enhanced Cohesiveness

Wet DCBF prepared as in Example 1 are rolled into a mass and kneaded to loosen any clumps of fibers and create more fiber entanglement throughout the mass. Once thoroughly kneaded, the whole mass is placed into the mold and the tissue is redistributed into the mold space by pressing with fingers or a spatula. The tissue is lyophilized in the mold, creating a shaped implant.

Molding DCBF in this manner results in an implant with enhanced cohesiveness when rehydrated, compared to an implant where tissue is placed into the mold in small chunks.

### Example 19: Preparation of a Shaped DCBF Implant with Enhanced Cohesiveness Using Syringe Mold

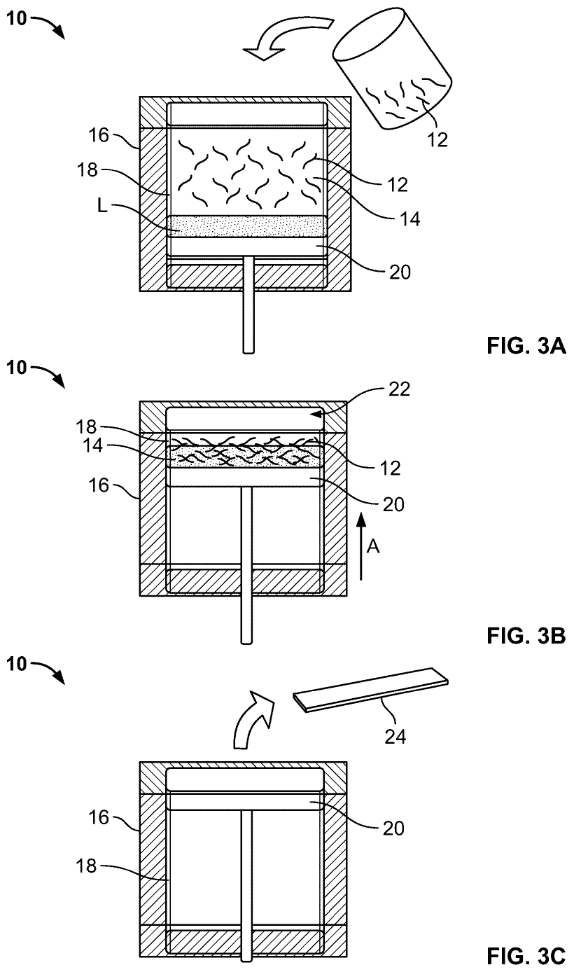

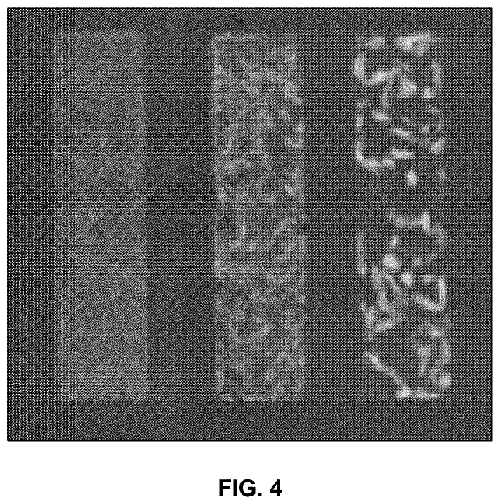

Use of a syringe mold to shape the DCBF is shown schematically in FIGS. 3A-3C and will now be described. As shown in FIG. 3A, wet DCBF (comprising DCBF fibers 12) prepared as in Example 1 are placed into a custom rectangular syringe mold 10, with an excess amount of liquid L (e.g., saline) so that the DCBF fibers 12 are homogenously suspended in solution 14. More particularly, the custom syringe mold 10 includes a housing 16, the walls of which form a chamber 18, within which is a reciprocatingly movable plunger 20 that defines the bottom of the chamber 18. The wet DCBF and liquid are placed into the chamber 18 of the mold 10. When the plunger 20 is in its undepressed position (see FIG. 3A), the inner dimensions of the chamber 18 may be, for example without limitation, approximately 10 cm(L)×2.5 cm(W)×127 mm(H). In some embodiments, the cross-section of the chamber 18 is in the shape of a rectangle with rounded edges. In such embodiments, the plunger 20 has dimensions of approximately 10 cm(L)×2.5 cm(W)×20 mm(H) and is also in the shape of a rectangle with rounded edges to match the cross-sectional opening of the chamber 18. An o-ring (not shown per se) seals the interface between the plunger 20 and the chamber 18.

As shown in FIG. 3B, a detachable filter 22, which is liquid permeable, fits across the top of the housing 16 to enclose the top of the chamber 18. After the DCBF fibers 12 and liquid L are placed into the chamber 18, the detachable filter 22 is placed over the open surface of the chamber such that when the plunger 20 is depressed (in the direction of arrow A), excess liquid is able to escape from the chamber 18 through the filter 22, while the DCBF fibers 12 are retained within. With reference to FIG. 3C, the filter 22 may then be detached and the implant 24 comprising the DCBF fibers (not shown in the densely packed implant of FIG. 3C) is pushed out of the chamber. The implant 24 is then lyophilized for storage and rehydrated prior to implantation. The density of the implant 24 may be altered by changing the distance that the plunger 20 is pushed toward the filter 22. Implants molded in this manner have enhanced cohesiveness and better shape retention due to the more homogenous distribution and enhanced entanglement of DCBF fibers in the implants.

### Example 20: Preparation of a Radio-Opaque Shaped DCBF Implant with Mineralized Bone

Wet DCBF prepared as in Example 1 is combined with mineralized cortical or cancellous granules/powder, placed into a rectangular mold, and shaped into an implant having dimensions of approximately 10 cm(L)×2.5 cm(W)×7 mm(H). The implant is removed from the mold and placed in a UV containment chamber where it is exposed to 315-400 nm UVA radiation for a period of about 30 minutes at an intensity in a range of from about 4,000 μwatts/cm² to about 20,000 μwatts/cm² before being removed and lyophilized. The inclusion of mineralized cortical or cancellous in the implant imparts additional radio-opacity due to the added mineral content. This allows for improved visualization of the graft by certain imaging methodologies (e.g. x-ray) during or after implantation of the graft material.

### Example 21: Preparation of a Radio-Opaque Shaped DCBF Implant with Selective Remineralization

Wet DCBF prepared as in Example 1 are placed into a custom cylindrical syringe mold yielding an implant with final dimensions of approximately 13 mm in height and 29 mm in diameter. The implant is removed from the mold and placed in a UV containment chamber where it was exposed to 315-400 nm UVA radiation for a period of about 30 minutes at an intensity in a range of from about 4,000 μwatts/cm² to about 20,000 μwatts/cm². The UV irradiation cross-links the outer surface of the implant allowing the implant to retain its shape when submerged in a liquid solution.

To remineralize the implant, two solutions are prepared as follows. The first solution is composed of 0.55M calcium chloride in DI water and the second solution is composed of 0.5M sodium phosphate in DI water. The irradiated implant is fully submerged in an aliquot of the first solution for 30 minutes under gentle agitation. After 30 minutes, the first solution is removed and the implant is submerged in an aliquot of the second solution for 30 minutes under gentle agitation. This process of alternating solutions is repeated until a hard mineralized shell develops on the surface of the implant that imparts radio-opacity that is comparable to normal mineralized human bone and increased mechanical strength of the implant through the remineralization of DCBF. The bulk/interior of the implant may also be mineralized in a similar fashion of alternating soaks of calcium chloride and sodium phosphate by the use of increased agitation and forcing the solutions through the bulk of the implant using positive or negative pressure. The implant is then lyophilized for storage and rehydrated prior to implantation.

### Example 22: Preparation of a Shaped DCBF Implant with Enhanced Cohesiveness and Biological Properties

Wet DCBF prepared as in Example 1 are combined with minced, powdered, or fibrous periosteum, placed into a rectangular mold, and shaped into an implant having dimensions of approximately 10 cm(L)×2.5 cm(W)×7 mm(H). The implant is removed from the mold and placed in a UV containment chamber where it was exposed to 315-400 nm UVA radiation for a period of about 30 minutes at an intensity in a range of from about 4,000 μwatts/cm² to about 20,000 μwatts/cm² before being removed and lyophilized. The inclusion of periosteum in the implant imparts enhanced cohesiveness and irrigation resistance due to the putty-like nature of periosteum and enhanced biological properties due to the addition of growth factors endogenous to the periosteum membrane.

### Example 23: Preparation of a Shaped DCBF Implant with Enhanced Cohesiveness by Heating

Wet DCBF prepared and molded into a shaped implant as in Examples 18 and 19 are placed in a heated chamber (e.g., lyophilizer, incubator, gravity oven), with or without UV exposure, at temperatures of from about 24° C. to about 70° C., and allowed to incubate for a period of time of from about 10 minutes to about 24 hours. The heating process improves the cohesiveness of the implant and prevents the implant from dispersing when placed in a rehydrating solution (e.g., water, saline, blood). After heating, the implant is lyophilized for storage and rehydrated prior to implantation.

### Example 24: Preparation of a Shaped DCBF Implant with Enhanced Cohesiveness Using Syringe Mold Having a Perforated Plunger

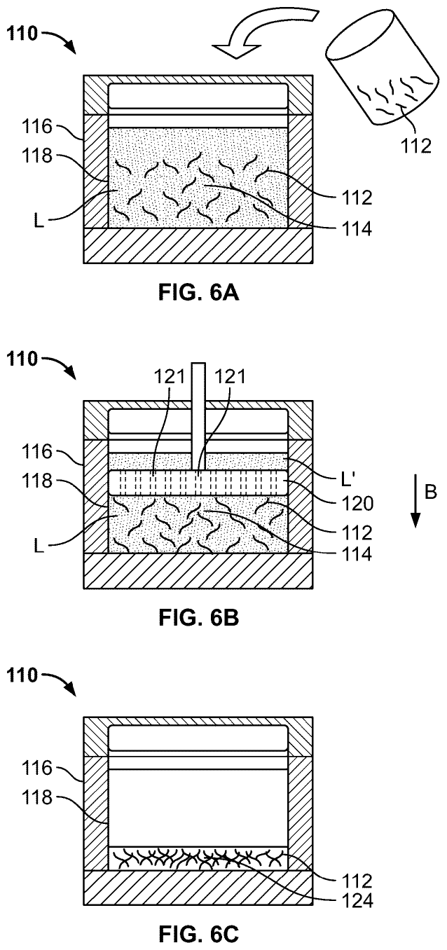

A different syringe mold than that used for Example 19 was used to shape DCBF fiber and is shown schematically in FIGS. 6A-6C. As shown in FIG. 6A, wet DCBF (comprising DCBF fibers 12) prepared as in Example 1 are placed into a custom rectangular syringe mold 110, with an excess amount of liquid L (e.g., saline) so that the DCBF fibers 112 are homogenously suspended in solution 114. More particularly, the custom syringe mold 110 includes a housing 116, the walls and bottom of which form a chamber 118 and within which is a reciprocatingly movable plunger 120 which is operated from the top of the mold and fits within the open cross section of the chamber 118. The wet DCBF 112 and liquid L are placed into the chamber 118 of the mold 110. Before the plunger 120 is depressed into the chamber 118 (see FIG. 6B), the inner dimensions of the chamber 118 may be, for example without limitation, approximately 10 cm(L)×2.5 cm(W)×127 mm(H). In some embodiments, the cross-section of the chamber 118 is in the shape of a rectangle with rounded edges. In such embodiments, as shown in FIG. 6B, the plunger 120 has dimensions of approximately 10 cm(L)×2.5 cm(W)×20 mm(H) and also has a cross sectional shape of a rectangle with rounded edges to match the cross-sectional opening of the chamber 118. An o-ring (not shown per se) seals the interface between the plunger 120 and the chamber 118. (The steps of the foregoing method are shown schematically in FIG. 5.)

As shown in FIG. 6B, the plunger has a plurality of perforations 121 for the movement of liquid L through the plunger, such that when the plunger is depressed within the chamber 118 in the direction shown by the arrow B, the plunger fits across the top of the housing 116, enclosing the chamber 118 and compressing the DCBF fibers 112. More particularly, after the DCBF fibers 112 and liquid L are placed into the chamber 118, the plunger 120 is depressed (in the direction of arrow B), excess liquid L′ is able to escape from the chamber 118 through the perforations 121, while the DCBF fibers 112 are retained and compressed within to form an implant. Each of the perforations 121 may be any suitable size and shape that will allow excess liquid to escape from the chamber 118, while retaining the DCBF fibers 112. For example, in some embodiments, without limitation, each perforation may have a circular shape with a diameter of about 1 millimeter, or about 1.5 millimeters, or even 2 millimeters. With reference to FIG. 6C, after the DCBF fibers have been compressed to the desired degree within the chamber 118, the excess liquid is poured out of the mold 110 and the plunger 120 removed. The height and density of the implant 124 may be altered by changing the distance that the plunger 120 is depressed in the chamber 118. In an embodiment, the implant 124 (still in the mold 110) is then frozen in a freezer at from about −80° C. to about 0° C. for up to 48 hours. The implant 124 (still in the mold 110) is then cured by warming using a warming plate or warm air at about 40° C. for about 8 hours. The implant 124 comprising the compressed treated DCBF fibers 112 (still in the mold 110) is lyophilized, after which the implant is easily removed from the chamber 118. After lyophilizing, the implant 124 may be stored and is rehydrated prior to implantation. Implants produced in this manner have enhanced cohesiveness and retain their shape better upon rehydration prior to use.

### Example 25: Determining the Wettability of the Shaped DCBF Implant

Produced Using Syringe Molds Having a Perforated Plunger Various implants produced by according to Example 24 were tested for wettability as follows and the results are provided below in Table 1. Differently shaped implants were tested where “bricks” were implants having a generally rectangular cross section, and “half pipes” were implants having a generally “C” shaped cross section.
1. Wettability Evaluation: A. drop of 0.9% saline solution (roughly 0.3 cc) is added to the top or bottom surface of the implant and the length of time it takes for each droplet to absorb into the implant is recorded.
2. Rehydration Evaluation: The implant is placed into a basin containing excess 0.9% saline solution to completely submerge the implant. The length of time it takes for the implant to absorb enough liquid to sink to the bottom of the basin is recorded.
3. Wettability/rehydration times utilizing the droplet test range from a few seconds (0:07) to a few minutes (4:00) whereas complete rehydration of the implant varies from 24 seconds to 20 minutes.
4. Preferred/suitable wettability/rehydration times for DCBF implants may be tailored to the end user/application and could range anywhere from near instantaneous wettability/rehydration (1-2 seconds) to much longer rehydration times of upwards of 20-30 min (preferably within 5 min).

TABLE 1

| | | | Complete |
|---|---|---|---|
| | Wettability time - | Wettability time- | rehydration - |
| | top surface | bottom surface | submerged |
| Implant Type | (mm:ss) | (mm:ss) | (mm:ss) |
| Brick - A | 1:15 | 1:02 | 2:50 |
| Brick - B | 0:15 | 0:15 | 0:24 |
| Brick - C | 1:18 | 1:16 | 2:40 |
| Brick - D | 0:45 | 0:07 | 1:15 |
| Brick - E | 2:00 | 3:00 | 20:00 |
| Brick - F | 0:38 | 0:31 | 2:15 |
| Brick - G | 0:24 | 1:03 | 3:20 |
| Brick - H | 0:29 | 1:05 | 13:00 |
| Half Pipe - A | 1:45 | 4:00 | 4:00 |
| Half Pipe - B | 0:45 | 1:09 | 2:50 |

### Example 26: Testing Shaped DCBF Implant Produced Using Syringe Molds Having a Perforated Plunger for Uniform Density

Various implants produced by according to Example 24 were tested for uniform densitys follows and the results are provided below in Table 2. Differently shaped implants were tested where a “disc” was an implant having a generally circular cross section, and “strips” were implants having a generally rectangular shaped cross section.
1. The weight of a DCBF implant was measured using an analytical balance.
2. Calipers were used to measure the dimensions of the implant, and the volume of the implant was calculated.
3. The density of the entire implant was calculated by dividing the weight recorded in step 1 by the volume calculated in step 2.
4. The implant was then cut into four equally sized pieces (quarters Q1, Q2, Q3 and Q4) using a scalpel. Steps 1-3 were repeated for each piece of the implant.
5. The relative standard deviation of the densities was calculated by dividing the standard deviation of the densities by the average. An implant can be said to have uniform density if the relative standard deviation of the measured densities is less than about 30%.

TABLE 2

| | Weight | Length | Width | Height | Volume | Density |
|---|---|---|---|---|---|---|
| Shape | (g) | (mm) | (mm) | (mm) | (cc) | (g/cc) |
| 5 cc disc | 1.317 | 44.81 | | 9.18 | 14.470 | 0.091 |
| Q1 | 0.349 | | | | 3.617 | 0.096 |
| Q2 | 0.319 | | | | 3.617 | 0.088 |
| Q3 | 0.308 | | | | 3.617 | 0.085 |
| Q4 | 0.292 | | | | 3.617 | 0.081 |
| | | | | | Average | 0.088 |
| | | | | | Std Dev | 0.005 |
| | | | | | % RSD | 6.028 |
| 20 cm strip | 2.823 | 192 | 9.16 | 7.25 | 12.751 | 0.221 |
| Q1 | 0.749 | 47.53 | 9.05 | 7.32 | 3.149 | 0.238 |
| Q2 | 0.582 | 47.15 | 9.17 | 6.29 | 2.720 | 0.214 |
| Q3 | 0.753 | 50.3 | 9.16 | 6.89 | 3.175 | 0.237 |
| Q4 | 0.754 | 48.94 | 9.18 | 7.05 | 3.167 | 0.238 |
| | | | | | Average | 0.230 |
| | | | | | Std Dev | 0.010 |
| | | | | | % RSD | 4.389 |
| 10 cm strip | 1.63 | 95.32 | 9.01 | 7.54 | 6.476 | 0.252 |
| Q1 | 0.386 | 24.05 | 9.03 | 7.65 | 1.661 | 0.232 |
| Q2 | 0.432 | 23.84 | 8.98 | 8.84 | 1.892 | 0.228 |
| Q3 | 0.386 | 25.06 | 8.92 | 6.77 | 1.513 | 0.255 |
| Q4 | 0.416 | 23.72 | 9.12 | 7.44 | 1.609 | 0.258 |
| | | | | | Average | 0.245 |
| | | | | | Std Dev | 0.012 |
| | | | | | % RSD | 5.055 |

While the disclosed invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the described invention.

We claim:

1. A method for producing an implant for treating a defect in bone and comprising demineralized cortical bone fibers (DCBFs) and mineralized bone pieces or particles, said method comprising the steps of:
combining and mixing DCBFs and mineralized bone pieces with a salt solution to produce mixed DCBFs and mineralized bone pieces in the salt solution;
soaking the mixed DCBFs and mineralized bone pieces in the salt solution for a period of time;
dehydrating the mixed DCBFs and mineralized bone pieces;
placing a quantity of the dehydrated mixed DCBFs and mineralized bone pieces and a chemical solution, which may or may not be the same as the salt solution, in a container having a shape; and
lyophilizing the mixed DCBFs and mineralized bone pieces in the container to form the implant.

2. The method of claim 1, wherein the implant has a first shape which is the shape of the container, is a least partially radiopaque, has a complete rehydration time of less than about 30 minutes, and one or more additional properties selected from:
a wettability time of less than about 5 minutes,
greater resistance to compression by an external force than an implant having equivalent mass and lacking mineralized bone pieces or particles, and
upon hydration with a biocompatible fluid, the implant forms a reshapable putty capable of being reshaped from the first shape to a desired second shape which is maintained until reshaped by external force or pressure.

3. The method of claim 1, wherein the chemical solution is selected from: a biocompatible polar molecule, a buffer solution, and combinations thereof.

4. The method of claim 1, wherein the weight ratio of mineralized bone pieces to DCBFs is in a range of from about 1:0.1 to about 0.1:1, based on air-dried weight.

5. The method of claim 1, further comprising the step of curing the implant at a temperature of from about 20° C. to about 50° C. for a period of up to about 48 hours by one or more techniques selected from air drying, heat drying and applying energy to the implant, said curing step being performed prior to said lyophilizing step, wherein the step of curing is performed prior to the step of lyophilizing, and after the step of placing the quantity of the dehydrated mixed DCBFs and mineralized bone pieces and the chemical solution in the container.

6. The method of claim 1, further comprising the step of crosslinking at least a portion of the implant by one or more techniques selected from: exposing the implant to energy, chemical crosslinking and heating, said crosslinking step being performed prior to said lyophilizing step.

7. The method of claim 6, wherein said crosslinking step is performed by exposing the implant to energy and the energy is selected from ultraviolet light or microwave energy.

8. The method of claim 1, further comprising, prior to lyophilizing, separating a portion of the chemical solution from the mixed DCBFs and mineralized bone pieces; and removing the liquid from the container.

9. The method of claim 8, wherein the step of shaping further comprises agitating the solution in the container to homogenously distribute the mixed DCBFs and mineralized bone pieces in the chemical solution.

10. The method of claim 9, wherein the container is a press mold comprising a reservoir for holding the solution therein and a plunger which is reciprocatingly movable in and out of the reservoir, wherein the step of separating is performed by pressing the plunger into the reservoir, thereby compressing the mixed DCBFs and mineralized bone pieces and causing the liquid to separate from the mixed DCBFs and mineralized bone pieces, by either flowing through a filter at an end of the reservoir that is opposite the plunger, or by flowing through a plurality of perforations provided in the plunger.

11. An implant for treating a defect in bone and which comprises demineralized cortical bone fibers (DCBFs) and mineralized bone pieces, wherein the DCBFs and mineralized bone pieces have been contacted/soaked with a salt solution prior to shaping and lyophilizing, wherein the implant is at least partially radiopaque and has a first shape, a residual moisture content of less than about 6% by weight, based on the total weight of the implant, and a complete rehydration time of less than about 30 minutes, and wherein said implant remains cohesive and retains its shape upon complete rehydration.

12. The implant of claim 11, further comprising one or more additional properties selected from:
a wettability time of less than about 5 minutes,
greater resistance to compression by an external force than an implant having equivalent mass and lacking mineralized bone pieces or particles, and
upon complete hydration with a biocompatible fluid, the implant forms a cohesive reshapable putty which is capable of being reshaped from the first shape to a desired second shape which is maintained until reshaped by external force or pressure.

13. The method of claim 11, wherein the weight ratio of mineralized bone pieces to DCBFs is in a range of from about 1:0.1 to about 0.1:1, based on air-dried weight.

14. The implant of claim 11, wherein the implant has uniform density.

15. The implant of claim 11, wherein the mineralized bone pieces comprise one or more of:
mineralized cortical bone fibers, cortical bone particles, cortical bone pieces, cancellous bone fibers, cancellous bone particles, cancellous bone pieces,

16. The implant of claim 11, further comprising one or more additional components selected from: cartilage fibers, cartilage particles, cartilage pieces, placental tissue, and periosteal tissue.

17. The implant of claim 16, wherein said placental tissue comprises amnion, chorion, umbilical cord.
